(19) United States
(12) Patent Application Publication
(10) Pub. No.: US 2020/0022331 A1
(43) Pub. Date: Jan. 23, 2020

(54) PET LITTER BOX

(71) Applicant: DEOCUBE, Seoul (KR)

(72) Inventor: Chang Hwan AHN, Seoul (KR)

(73) Assignee: DEOCUBE, Seoul (KR)

(21) Appl. No.: 16/496,040

(22) PCT Filed: Aug. 12, 2017
(86) PCT No.: PCT/KR2017/008811

§ 371 Date: Sep. 20, 2019

(30) Foreign Application Priority Data
Mar. 22, 2017 (KR) 10-2017-0036355

(51) Int. Cl. A01K 1/01; A01K 1/00; A61L 2/10; A61L 9/014

(52) U.S. Cl. CPC A01K 1/0107; A01K 1/0064; A61L 2/10; A61L 2209/22; A61L 2202/11; A61L 2209/14; A61L 9/014

(57) ABSTRACT

The present invention relates to a pet litter box which prevents the surrounding environment from becoming contaminated due to excrement of a pet being exposed to the outside, comprises a main body, a cover, a door, and a multifunctional filter unit which are separably coupled, so as to easily prevent the generation of odor from the excrement of a pet and bacterial growth, and can prevent unnecessary expense increases while achieving usage convenience. The pet litter box comprises: a main body having a box shape of which an upper portion is open; a cover which is formed to be coupled to the main body so as to cover the open portion of the main body, and which is provided with a pet entrance at the front thereof, open windows on opposing left and right sides thereof to communicate with the outside, and a vent window in an upper portion thereof which forms a connecting part of the open windows, wherein a door is coupled to the pet entrance so as to be openable and closable, and a multifunctional filter unit, which has antibacterial, and odor adsorption and elimination functions, is detachably coupled to the open windows and the vent window.

### TECHNICAL FIELD

The present invention relates to a pet litter box, and more particularly, to a pet litter box to prevent surrounding environments from being contaminated due to pet excretion exposed to the outside, and implement a convenient use.

### BACKGROUND ART

A pet refers to an animal raised with affection by a human and including various animals such as dogs, cats, and birds.

The pet may give emotional stability or pleasure to humans. There is a growing number of dog lovers that raise dogs and cat lovers that raise cats, and various animals in addition to the dogs and cats are sharing living places with humans.

For example, conventionally, when intending to raise a pet, humans generally have raised the pet after preparing a separate space outside a house. Recently, dogs, cats or the like generally live together in an indoor space in which the humans live.

Accordingly, problems of treating excretion of pets living together in human living environments continuously occur. The separation and disposal of the pet excretion without contaminating indoor environments has become an important problem for humans living with the pets.

To solve this, a method of providing an excretion mat or the like on which a pet defecates at a certain place has been disclosed in the related art as shown in Document 1.

The conventional method of providing an excretion mat such as Korean Utility Model Registration No. 20-0291041 (Sep. 19, 2002) enables the pet to defecate on the excretion mat when the pet feels defecation, but the excreting process of the pet and the excretion are exposed to the outside and thus the indoor environments may be easily contaminated due to the excretion and the odor of the excretion.

Accordingly, pet litter boxes are recently disclosed in Korean Utility Model Registration No. 20-0468228 (Jul. 25, 2013), and Korean Patent Registration No. 10-1477320 (Dec. 22, 2014) to prevent the excretion of the pet from being exposed to the outside.

However, the pet litter boxes in the related art such as Korean Utility Model Registration No. 20-0468228 (Jul. 25, 2013), and Korean Patent Registration No. 10-1477320 (Dec. 22, 2014) may achieve the function of preventing the pet excretion from being disposed to the outside, but fail to suppress the external exposure of the odor of the excretion and various harmful bacteria, and the problem that the indoor environments are contaminated continuously occurs due to the odor of the excretion upon a long-term use.

### DETAILED DESCRIPTION OF THE INVENTION

### Technical Problem

In order to solve the above problems in the related art, objects of the present invention are as follows.

First, the pet excretion is prevented from being exposed to the outside so that the indoor environments can be prevented from being contaminated due to the pet excretion even when the pet litter box is used for a long time.

Second, a multifunctional filter, which has an odor adsorption and decomposition function and an antibacterial function to prevent the odor and bacteria generated from the pet excretion from spreading, is provided so that contamination and odor caused by the pet excretion can be minimized.

Third, a main body, a cover, a door, and a multifunctional filter unit are detachably configured so that the pet litter box can be installed after conveniently carried to a place desired to be installed, and only the damaged part may be easily replaced when either configuration is damaged during use so that a stable use can be implemented.

Fourth, the multifunction filter provided on the cover can be prevented from being damaged by claws or teeth of the pet, and the stable and efficient use can be implemented.

Fifth, ultraviolet rays may be generated at a place without light or at night through an antibacterial lamp provided inside the cover, so that the odor decomposition function of the multifunctional filter can be promoted, and the antibacterial effect on the pet excretion located inside the main body can be improved.

### Technical Solution

The pet litter box according to the present invention for achieving the above objects includes: a box-shaped main body having an opened upper portion; and a cover coupled to cover the opened upper portion of the main body, formed at a front side thereof with a pet entrance, formed at opposing left and right sides thereof with open windows communicating with an outside, and formed with a vent window at an upper portion forming a connection part between the open windows, wherein a door is coupled to the pet entrance so as to be opened and closed, and a multifunctional filter unit having an odor adsorption and decomposition function and an antibacterial function is detachably coupled to the open windows and the vent window.

The main body may include a plurality of locking portions formed along an edge adjacent to the opened portion of the main body, the cover may include a plurality of coupling portions at positions corresponding to the locking portions, and the locking portion and the coupling portions may be coupled to each other through fastening devices, respectively.

In addition, the door may include a door connection frame detachably coupled to the pet entrance of the cover, and a door body coupled to the door connection frame so as to be opened and closed, in which the door body may include a first door hinged to the door connection frame, a second door detachably coupled to the first door, and a multifunctional filter provided between the first door and the second door.

In addition, the multifunctional filter unit may include a fixing frame formed therein with a plurality of through holes corresponding to the open windows and the vent window of the cover so as to be detachably coupled to the cover, and a multifunctional filter provided inside the fixing frame to cover the open windows and the vent window.

In addition, the body may include at least one excretion shovel storage groove for storing an excretion shovel for removing the pet excretion.

In addition, at least one antibacterial lamp may be detachably provided inside the cover.

In addition, a plurality of anti-slip packings may be further provided at a lower end of the main body.

Meanwhile, a plurality of fastening protrusions may be provided at a lower end of the fixing frame, the cover may be formed with a plurality of coupling holes at positions corresponding to the fastening protrusions, and the multifunctional filter may be formed with a plurality of connection holes which the fastening protrusions pass through and are coupled to, respectively.

### Advantageous Effects of the Invention

Advantageous effects according to the above-described present invention are as follows.

First, the excretion of the pet may be prevented from being exposed to the outside, and the odor of the pet excretion may be suppressed through the provided multi-functional filter, so that the odor due to the pet excretion stacked on the main body can be prevented from being generated even when the pet litter box is used for a long time, and the indoor environments can be prevented from being contaminated.

Second, the main body, the cover, the door, and the multifunctional filter unit are detachably provided so that the pet litter box can be installed after easily carried to a place desired to be installed, and only the damaged part may be easily replaced when either configuration is damaged during use so that the stable use can be implemented. Thus, unnecessary costs can be prevented from increasing in addition to the convenience in use and the stable use can be implemented.

Third, the multifunction filters provided on the main body and the cover can be prevented from being damaged by claws or teeth of the pet, and stable and sustainable use can be implemented so that efficient use can be implemented without replacing unnecessary equipment despite long time use.

Fourth, the configuration such as the antibacterial lamp provided in the cover, the excretion shovel storage groove provided in the main body, and the anti-slip packing can increase the convenience in use.

### DESCRIPTION OF THE DRAWINGS

FIG. 1 is an exploded perspective view showing a pet litter box according to the present invention.

FIG. 2 is a combined perspective view showing the pet litter box according to the present invention.

FIG. 3 is a front view showing the pet litter box according to the present invention.

FIG. 4 is a top view showing the pet litter box according to the present invention.

FIG. 5 is a sectional view taken along line A-A of FIG. 4.

FIG. 6 is an exploded main parts extraction perspective view showing a coupling state between a cover and a multifunctional filter unit according to the present invention.

### BEST MODE

The terms and words used in the specification and claims of the present invention should not be construed as limited to a conventional or lexical meaning, and should be construed as the meanings and concepts consistent with the technical idea of the present invention based on the principle that “an inventor may define the concept of the term properly in order to describe the invention in the best way”.

In addition, the embodiments described in the present specification and the configurations shown in the drawings are merely preferred embodiments according to the present invention, and do not represent all of the technical ideas of the present invention. Therefore, it should be understood that various equivalents and modifications may be substituted therefor at the time of filing of the present application.

The present invention is configured to include a box-shaped main body 10 having an opened upper portion; and a cover 20 coupled to cover the opened upper portion of the main body 10, formed at a front side thereof with a pet entrance 21, formed at opposing left and right sides thereof with open windows 22 communicating with an outside, and formed with a vent window 23 at an upper portion forming a connection part between the open windows 22, wherein a door 30 is coupled to the pet entrance 21 so as to be opened and closed, and a multifunctional filter unit 40 is detachably coupled to the open windows 22 and the vent window 23.

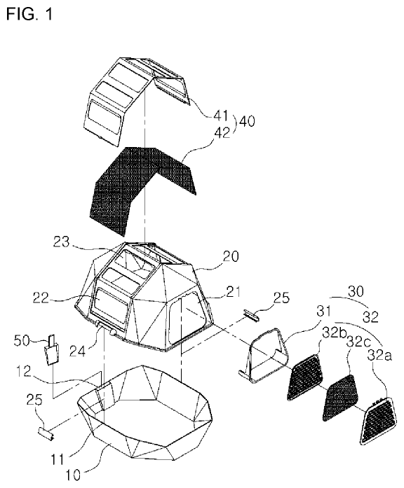

As shown in FIG. 1, the main body 10 may be formed in a box shape to have an opened upper portion, and the main box may have a box shape having a flat bottom surface so as to be installed at a desired position for the pet litter box and to firmly maintain the position.

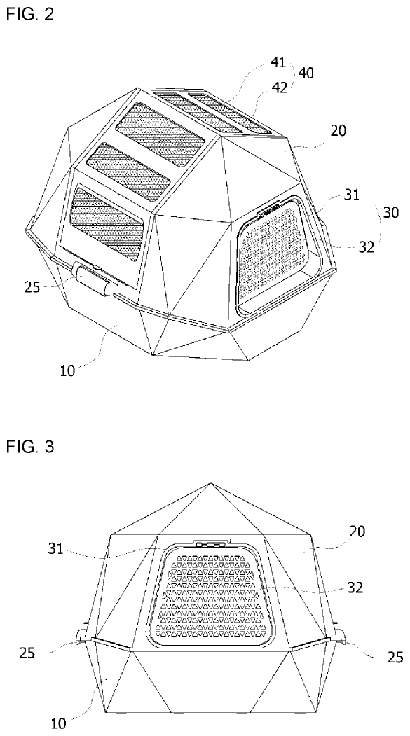

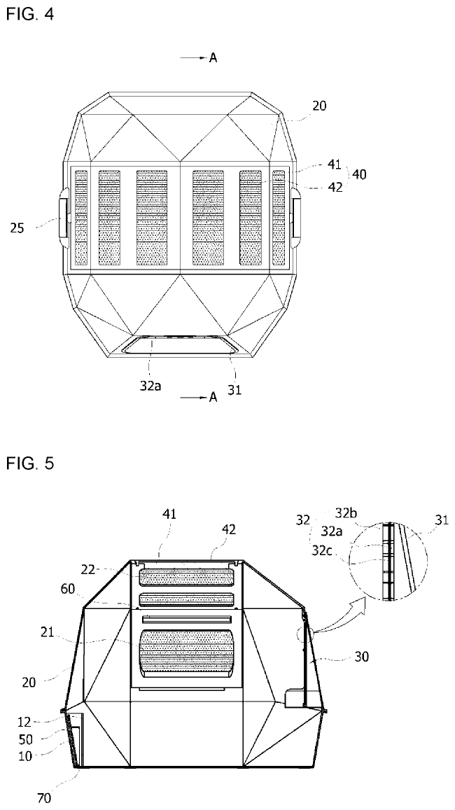

In addition, as shown in FIGS. 1 to 4, the main body 10 may have an external contour formed in a polygonal shape, in which the contour intends to increase an interior effect according to the installation position.

In addition, as shown in FIG. 1, the cover 20 is configured to be coupled to the opened portion of the main body 10 while surrounding the opened portion of the main body 10. In order to achieve the above configuration, the main body 10 may be formed with a plurality of locking portions 11 along an edge adjacent to the opened portion, the cover 20 may be formed with a plurality of coupling portions 24 at positions corresponding to the locking portions 11, and the locking portion 11 and the coupling portions 24 may be detachably coupled to each other through fastening devices 25, respectively.

Although the fastening devices 25 have been described as having a hook shape in the present invention, the fastening devices 25 are not limited thereto, and the fastening shapes and configurations may be modified in various forms according to the user's selection as long as the main body 10 is detachably connected to the cover 20.

In addition, the pet entrance 21 provided in the cover 20 may be formed to allow the pet to enter and exit the main body 10. A plurality of open windows 22 and the vent window 23 formed in the cover 20 may be formed in a hole shape at an upper portion of the cover 20.

As shown in FIGS. 1 to 3, the door 30 coupled to the pet entrance 21 may include a door connection frame 31 detachably coupled to the pet entrance 21 of the cover 20, and a door body 32 coupled to the door connection frame 31 so as to be opened and closed.

In particular, the door body 32 may include a first door 32a hinged to the door connection frame 31, a second door 32b detachably coupled to the first door 32a, and a multifunctional filter 32c provided between the first door 32a and the second door 32b.

More specifically, the door connection frame 31, as shown in FIG. 1, may be detachably coupled to the pet entrance 21 of the cover 20, and a door body 32 may be rotatably connected to one side of the door connection frame 31, in which the door body 32 may be connected to the door connection frame 31 so as to be easily rotated around a connection portion with respect to the door connection frame 31 when the pet exerts a predetermined force on the door body 32.

Further, the multifunctional filter 32c is configured to be interposed between the first door 32a hinged to the door connection frame 31 and the second door 32b detachably coupled to the first door 32a in the door body 32 as described above, so that bacteria from the excretion can be prevented from being generated and the odor can be prevented from spreading even when the pet excretion is stacked inside the main body 10.

In particular, the multifunctional filter 32c may be positioned between the first door 32a and the second door 32b, so that the multifunctional filter 32c can be prevented from being easily damaged when the pet enters and exits the main body 10 through the door body 32 or the pet scratches the door body 32 using teeth or claws.

In addition, the multifunctional filter unit 32c may have through holes having both the odor adsorption and decomposition function and the antibacterial function as shown in FIG. 1, so that the odor of the pet excretion can be removed, the bacteria can be suppressed, and the air circulation between the main body 10 and the outside can be easily implemented.

In addition, as shown in FIG. 1, the multifunctional filter unit 40 installed on an upper side of the cover 20 may include a fixing frame 41 formed therein with a plurality of through holes 41a corresponding to the open windows 22 and the vent window 23 of the cover 20 so as to be detachably coupled to the cover 20, and a multifunctional filter 42 provided inside the fixing frame 41 to cover the open windows 22 and the vent window 23.

The fixing frame 41 is configured to facilitate the installation of the multifunctional filter 42 to the upper side of the cover 20. The multifunctional filter 42, as shown in FIGS. 1 to 5, may be coupled by the fixing frame 41 to suppress an external exposure while covering the open windows 22 and the vent window 23 of the cover 20, so that the multifunctional filter 42 can be prevented from being easily damaged even when the pet scratches the multifunctional filter 42 using teeth or claws at an inside and outside of the cover 20.

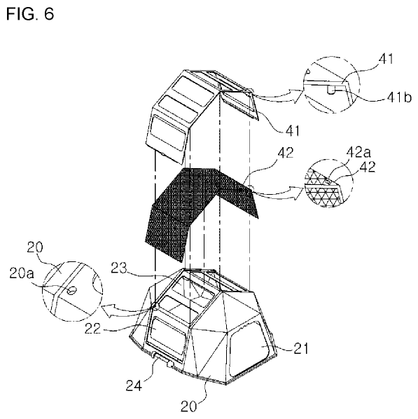

In addition, as shown in FIG. 6, the fixing frame 41 may be provided at a lower end thereof with a plurality of fastening protrusions 41b, the cover 20 may be formed with a plurality of coupling holes 20a at positions corresponding to the fastening protrusions 41a, and the multifunctional filter 42 is formed with a plurality of connection holes 42a which the fastening protrusions 41b pass through and are coupled to, respectively. Thus, when the multifunctional filter 42 is coupled between the fixing frame 41 and the main body 20, the coupling and separation may be easily implemented, and a position of the multifunctional filter 42 coupled between the fixing frame 41 and the main body 20 may be firmly maintained after the mutual coupling.

The configuration and function of the multifunctional filter 42 may be the same as the configuration and function of the above-described multifunctional filter 32c.

Although the present invention has been described that the multifunctional filter 32c may be provided between the first door 32a and the second door 32b of the door body 32, and the multifunctional filter 42 may be provided between the open windows 22 and vent window 23 of the cover 20 and the fixing frame 41, the multifunctional filter 32c may be inserted between the door body 32, or the multifunctional filter 42 is inserted between the fixing frame 41, and the configuration may be variously changed and applied according to user's choice as long as the external exposure of the multifunctional filters 32c and 42 is suppressed and the installed multifunctional filters 32c and 42 are prevented from being damaged by the teeth or claws of the pet.

In addition, the main body 10 may include at least one excretion shovel storage groove 12 for storing an excretion shovel 50 for removing the pet excretion.

As shown in FIGS. 1 to 5, the excretion shovel storage groove 12 may be formed inside the main body 10, or may be formed on an outer side of the main body 10 according to user's choice.

In addition, at least one antibacterial lamp 60 may be detachably provided inside the cover 20.

The antibacterial lamp 60 may generate ultraviolet rays at a place without light or at night to promote the odor decomposition function of the multifunctional filter 32c and 42, thereby eliminating the bacteria from the pet excretion even when the pet excretion is stacked inside the main body 10, and preventing the body 10 from being contaminated due to the pet excretion or the indoor environment from being contaminated due to the contamination of the main body 10.

In addition, the antimicrobial lamp 60 may be properly installed and used after the user selects one of an ultraviolet lamp or a fluorescent lamp.

Although not shown in detail in the present invention, the antibacterial lamp 60 may be detachably provided to the inside of the cover 20 through a fastening tool such as a bolt (not shown) and a nut (not shown).

In addition, a plurality of anti-slip packings 70 may be provided at a lower end of the main body 10, so that the pet litter box can be prevented from moving or shaking and falling down from the installed position when the pet enters or exits the main body 10 or a shock is transmitted to the pet litter box due to a movement of the pet in a state where the pet litter box of the present invention is installed.

As described above, when the pet litter box according to the present invention is installed and used indoors as a pet toilet, the indoor environments can be prevented from being contaminated due to the pet excretion, and the maintenance and repair can be easily implemented during use, so that efficient use can be implemented without unnecessary expense.

Although the present invention has been shown and described with respect to particular embodiments, it will be apparent to a person having ordinary skill in the art that various deformations and modifications are available within the scope without departing from the spirit and scope of the present invention.

1. A pet litter box comprising:
a box-shaped main body (10) having an opened upper portion; and
a cover (20) coupled to cover the opened upper portion of the main body (10), formed at a front side thereof with a pet entrance (21), formed at opposing left and right sides thereof with open windows (22) communicating with an outside, and formed with a vent window (23) at an upper portion forming a connection part between the open windows (22), wherein
a door (30) is coupled to the pet entrance (21) so as to be opened and closed, and a multifunctional filter unit (40) having an odor adsorption and decomposition function and an antibacterial function is detachably coupled to the open windows (22) and the vent window (23), and wherein
the multifunctional filter unit (40) includes a fixing frame (41) formed therein with a plurality of through holes (41a) corresponding to the open windows (22) and the vent window (23) of the cover (20) so as to be detachably coupled to the cover (20), and a multifunctional filter (42) provided inside the fixing frame (41) to cover the open windows (22) and the vent window (23).

2. The pet litter box of claim 1, wherein the main body (10) includes a plurality of locking portions (11) formed along an edge adjacent to the opened portion of the main body (10), the cover (20) includes a plurality of coupling portions (24) at positions corresponding to the locking portions (11), and the locking portion (11) and the coupling portions (24) are coupled to each other through fastening devices (25), respectively.

3. The pet litter box of claim 1, wherein the door (30) includes a door connection frame (31) detachably coupled to the pet entrance (21) of the cover (20), and a door body (32) coupled to the door connection frame (31) so as to be opened and closed, and wherein
the door body (32) includes a first door (32a) hinged to the door connection frame (31), a second door (32b) detachably coupled to the first door (32a), and a multifunctional filter (32c) provided between the first door (32a) and the second door (32b).

4. The pet litter box of claim 1, wherein at least one detachable antibacterial lamp (60) is provided inside the cover (20).

5. The pet litter box of claim 1, wherein the fixing frame (41) is provided at a lower end thereof with a plurality of fastening protrusions (41b), the cover (20) is formed with a plurality of coupling holes (20a) at positions corresponding to the fastening protrusions (41b), and the multifunctional filter (42) is formed with a plurality of connection holes (42a) which the fastening protrusions (41b) pass through and are coupled to, respectively.
